(19) United States
(12) Patent Application Publication
(10) Pub. No.: US 2024/0165170 A1
(43) Pub. Date: May 23, 2024

(54) USE OF LACTIC ACID BACTERIUM SET FOR PREVENTING OR TREATING NSAID-INDUCED GASTRIC ULCER

(71) Applicant: GRAPE KING BIO LTD., Taoyuan City (TW)

(72) Inventors: Chin-Chu CHEN, Taoyuan City (TW); Yen-Lien CHEN, Taoyuan City (TW); Shin-Wei LIN, Taoyuan City (TW); You-Shan TSAI, Taoyuan City (TW); Ci-Sian WANG, Taoyuan City (TW); Yu-Hsin HOU, Taoyuan City (TW); Yang-Tzu SHIH, Taoyuan City (TW); Ching-Wen LIN, Taoyuan City (TW); Ya-Jyun CHEN, Taoyuan City (TW); Jia-Lin JIANG, Taoyuan City (TW); Zi-He WU, Taoyuan City (TW); Yen-Po CHEN, Taoyuan City (TW); Tzu Chun LIN, Taoyuan City (TW)

(73) Assignee: GRAPE KING BIO LTD., Taoyuan City (TW)

(21) Appl. No.: 18/117,573

(22) Filed: Mar. 6, 2023

(30) Foreign Application Priority Data
Nov. 21, 2022 (TW) 111144439

(51) Int. Cl. A61K 35/744; A23L 33/00; A23L 33/135; A61K 35/747; A61P 1/04; C12N 1/20

(52) U.S. Cl. CPC A61K 35/744; A23L 33/135; A23L 33/40; A61K 35/747; A61P 1/04; C12N 1/20; A61K 2035/115

(57) ABSTRACT

The present invention provides a method for preventing and/or treating a NSAID-induced gastric ulcer. The method comprises administrating an effective amount of a lactic acid bacterium set to a subject. The lactic acid bacterium set comprises Lactobacillus plantarum GKD7 and Pediococcus acidilactici GKA4.

### BACKGROUND OF THE INVENTION

### Field of the Invention

The present invention relates to a use of a lactic acid bacterium set and, in particular, to a use of a lactic acid bacterium set comprising Lactobacillus plantarum GKD7 and Pediococcus acidilactici GKA4, which can prevent or treat NSAID-induced gastric ulcer.

### Description of the Prior Art

Gastric injury includes gastroesophageal reflux, gastric ulcer and Mar-Wei syndromes, wherein gastric ulcer (GU) generally refers to ulcer lesions in gastric mucosa.

Common symptoms of gastric ulcer include hiccups, vomiting, pain, poor appetite and unexplained weight loss. If it is not treated immediately, the symptoms will easily develop into serious conditions such as gastric bleeding and perforation. The prevalence of gastric ulcer in Taiwan accounts for about 4.7% of the population (about 1 million people), which is more than twice that of Europe. The main factors that cause gastric ulcer include Helicobacter pylori and nonsteroid anti-inflammatory drugs (NSAIDs). The two factors cause gastric ulcer for different reasons.

Helicobacter pylori can attach to the mucous membrane of the digestive tract and produce ammonia by decomposing urea, thereby neutralizing gastric acid. This will cause an overly strong immune response at the infected site, increase the secretion of gastric acid, destroy the barrier structure of the mucosal cells, and finally cause gastric ulcer lesions.

NSAIDs include aspirin, Ibuprofen and Naproxen, etc., which are common analgesic, anti-inflammatory and antipyretic agents, of which aspirin is the most commonly used. Gastric ulcers caused by NSAIDs are particularly noteworthy because of their widespread use.

The medical functions of aspirin are antipyretic, analgesic and inhibition of platelet aggregation, etc., and its mechanism is mainly to achieve anti-inflammatory, analgesic and antithrombotic effects by inhibiting the action of cyclooxygenase (COX). In addition, aspirin can inhibit the synthesis of prostaglandin (PG) in the body to achieve antipyretic and analgesic effects. However, prostaglandin E1 (PGE1) has the function of inhibiting gastric acid secretion and enhancing the resistance of gastric mucosa, so the common side effect of aspirin is gastric ulcer caused by inhibiting PGE1.

Lactic acid bacteria have multiple benefits for human health, such as helping digestion and improving intestinal health. However, there is currently no relevant research on the therapeutic effect of lactic acid bacteria on gastric ulcer caused by NSAIDs.

### SUMMARY OF THE INVENTION

The present invention provides a use of a lactic acid bacterium set, which can be used to manufacture a composition for preventing and/or treating a gastric lesion. The lactic acid bacterium set comprises Lactobacillus plantarum GKD7 and Pediococcus acidilactici GKA4.

In one embodiment, Lactobacillus plantarum GKD7 is deposited in NITE Patent Microorganisms Depositary (NPME)), Japan, and the deposit number is NITE BP-03313.

In one embodiment, Pediococcus acidilactici GKA4 is deposited in NITE Patent Microorganisms Depositary (NPMD), Japan, and the deposit number is NITE BP-03311.

In one embodiment, a weight ratio of Lactobacillus plantarum GKD7 and Pediococcus acidilactici GKA4 in the lactic acid bacterium set is 1:1.

In one embodiment, the composition is administered to a subject in an effective amount of 0.05 g to 5 g/60 kg body weight/day.

In one embodiment, the gastric lesion includes gastroesophageal reflux, a gastric ulcer and Mar-Wei syndromes.

In one embodiment, the gastric lesion refers to necrosis or affection of gastric mucosal epithelial cells.

In one embodiment, compared to a subject not administered with the composition, a mucosal ulcer index of the subject administered with the composition is lower, thereby achieving the effect of preventing and/or treating the gastric lesion.

In one embodiment, the lactic acid bacterium set is prepared by the following method:
(a) inoculating bacteria of the lactic acid bacterium set into a solid-state medium for solid-state culture to form a colony;
(b) inoculating the colony cultivated in step (a) into a liquid medium for liquid culture to obtain a bacterial liquid of the liquid medium containing the bacteria;
(c) centrifuging the bacterial liquid of the liquid medium containing the bacteria in step (b) to obtain bacterial slime; and
(d) freeze-drying the bacteria slime obtained in step (c) as a freeze-dried powder;
wherein bacteria of Lactobacillus plantarum GKD7 and bacteria of Pediococcus acidilactici GKA4 can be cultured separately and then mixed.

In one embodiment, a freeze-drying temperature in step (d) is −196 to −40° C.

In one embodiment, the composition is a pharmaceutical composition, and the pharmaceutical composition further comprises a pharmaceutically acceptable carrier, an excipient, a diluent or an adjuvant.

In one embodiment, the composition is a food additive.

In one embodiment, an administration mode of the composition includes oral administration, drops and suppositories.

In order to make the above-mentioned and other aspects of the present invention clearer, the specific examples below are described in conjunction with the accompanying drawings.

### BRIEF DESCRIPTION OF THE DRAWINGS

FIG. 1 depicts the body weight change of 4 groups of mice during the test;

FIG. 2 depicts the ulcer area of 4 groups of mice after the end of the experiment;

FIG. 3 depicts the ulcer index of 4 groups of mice after the end of the experiment;

FIG. 4 depicts the results of the mucosal ulcer scores of the 4 groups of mice after the experiment.

### DETAILED DESCRIPTION OF THE EMBODIMENTS

### Culture of the Lactic Acid Bacterium Set

The Lactobacillus plantarum GKD7 used in this example has been deposited in Taiwan Foundation Bioresource Collection and Research Center (BCRC), and the deposit number is BCRC 910877. The strain is also deposited in Japan's international depositary authority: National Institute of Technology and Evaluation, Patent Microorganisms Depositary (NPMD), and the deposit number is NITE BP-03313.

Similarly, the Pediococcus acidilactici GKA4 used in this example has been deposited in Taiwan Foundation Bioresource Collection and Research Center, and the deposit number is BCRC 910876. The strain is also deposited in Japan's international depositary authority: National Institute of Technology and Evaluation, Patent Microorganisms Depositary, and the deposit number is NITE BP-03311.

It is particularly noted that both the above-mentioned lactic acid bacteria strains can be purchased on the market (for example, on the official website of BCRC, https://catalod.bcrc.firdi.ord.tw/) and can be readily available by those skilled in the art. Furthermore, as long as the lactic acid bacteria strains used in the present invention are GKD7 and GKA4, they are not limited to those obtained from the above deposit sources.

The colonies of Lactobacillus plantarum GKD7 and Pediococcus acidilactici GKA4 were respectively picked and inoculated on independent solid media to activate the strains. The solid medium can be, for example, MRS agar. After the bacteria grow vigorously, the fresh bacteria were transferred together with the solid medium into the Erlenmeyer flask containing the liquid medium for further liquid culture. The liquid culture can be cultured under the conditions of a temperature of 35 to 50° C., an aeration rate of 0 to 1 wm, an aerated gas of nitrogen or carbon dioxide, and a shaking rate of 10 to 100 rpm. The time of liquid culture can range from 16 to 24 hours, preferably 18 hours. The liquid medium is, for example, the MRS liquid medium in the following table.

Formulation of MRS liquid medium

| Composition | Proportion (weight percent) |
|---|---|
| Glucose | 1-10% |
| Yeast extract | 0.1-5% |
| Peptone | 0.1-5% |
| Trace elements | 0.01-2% |
| Cysteine | 0.01-0.1% |
| Tween-80 | 0.05-1% |
| Water | 76.9-98.73% |

### Preparation of Lyophilized Powder

After the growth of the bacteria in liquid culture was completed, the liquid culture medium containing the bacteria was collected and centrifuged to obtain the bacterial slime. The centrifugation rate was, for example, 1000 to 15000 rpm. The protective agent (6-30% skimmed milk powder) was added to the obtained bacteria slime, mixed and freeze-dried, and stored at low temperature after freeze-drying, which was the lactic acid bacteria used in the present invention. Preferably, the freeze-drying temperature was set at −196 to −40° C., which can avoid destroying the activity of the strain. The freeze-drying time was, for example, 16 to 72 hours. The storage temperature after freeze-drying can be −30° C. to 0° C.

### Experimental Animals

A total of 24 ICR male mice purchased from BioLASCO (Lesco Biotechnology) were used as the experimental animals, each about 25 grams. The mice were kept in a general mouse cage, the room temperature was maintained at 22±3° C., the humidity was 55±15%, and the light and darkness were regularly 12 hours (12 hr light-dark cycle). Feed and sterile reverse osmosis water was allowed to be eaten by mice freely. Newly imported animals need to be observed for 3-5 days to ensure that there is no abnormality before testing. Before the experiment, the ICR mice were randomly grouped and caged, and each ICR mouse was marked with an ear number for identification and recording.

### Experiment Design

24 mice were divided into a blank control group, a negative control group, a positive control group and a lactic acid bacteria test group, and there were 6 mice in each group. The mice were administered once a day for 39 consecutive days. The blank control group and the negative control group were given drinking water, the positive control group was given 20 mg/kg Omeprazole, and the lactic acid bacteria test group was given GKD7 and GKA4 freeze-dried powder, each 10.2 mg/kg/day. On day 29, 500 mg/kg aspirin was administered to the negative control group, positive control group and lactic acid bacteria test group to induce gastric ulcer (the blank control group was not administered) for 10 days (D29-D39), and finally the mice were sacrificed on D40 and performed various analyses.

In this experiment, Omeprazole administered to the positive control group is an oral drug that can be used to treat gastroesophageal reflux, gastric and duodenal ulcers and gastrinoma.

### Test Record and Analysis

The body weight of the mice was recorded every week, and after 39 consecutive days of administration, the mice were sacrificed with a high dose of Sutai 50 on the 40th day. The appearance, stomach tissues and organs of the mice were observed and made records.

Ulcer Analysis

Ulcer area statistics: ImageJ software was utilized to circle the ulcer focus of gastric tissue to calculate the gastric ulcer focus area of each ICR mouse. Each ulcer focus can get different scores according to the size of the ulcer (see the table below):

Ulcer Area Classification and Scoring

| Grading | Level I | Level II | Level III |
|---|---|---|---|
| Area size | <1 mm² | 1-3 mm² | >3 mm² |
| Score | 1 | 2 | 3 |

After each ulcer focus score is obtained, the application calculates the ulcer index of the mouse according to the following formula 1, and the higher the ulcer index, the more severe the ulcer.

$$Ulcer index ( UI ) = \qquad ( Formula 1 )$$

$$\frac{( 1 [ level I number ) + ( 2 \times level II number ) + ( 3 \times level III number )}{( Number of experimental mice in each group )}$$

If the experimental group mice (positive control group, lactic acid bacteria test group) are compared with the negative control group mice and the area of gastric ulcer (ulcer index) is reduced, it means that the experimental group produces a healing effect. Dividing the ulcer index of the experimental group by the ulcer index of the negative control group obtains the ulcer improvement rate, which is then subtracted from 1 to get the healing rate (cure rate) compared with the negative control group. The calculation method is shown as the following formula 2:

$$Cure rate ( \% ) = \qquad ( Formula 2 )$$

$$[ 1 - ( \frac{\begin{matrix}UI of positive control group / \\ UI of lactic acid bacteria test group\end{matrix}}{UI of negative control group} ) ] \times 100 \%$$

The higher the cure rate, the higher the degree of ulcer improvement in the group of mice.

Pathological analysis: pathological analysis was carried out from the mucosal ulcer index.

Mucosa ulcer: it represents the severity of ulcer focus due to gastric mucosal epithelial cell necrosis caused by aspirin. Grade 0 represents no ulcers occurred in normal tissue; Grade 1 represents less than ⅓ of superficial ulcer lesions in gastric mucosa; Grade 2 represents about ½ of superficial to middle ulcer lesions in gastric mucosa; Grade 3 represents about ⅔ of superficial to deep ulcer lesions in gastric mucosa; and grade 4 represents gastric mucosal epithelial full-thickness ulcer and the lesion penetrates 3/3 of the muscle layer.

Statistical Analysis

The data obtained in this experiment were analyzed with Graphpad Prism 6 statistical analysis software for one-way/two-way analysis of variance, and p<0.05 indicated a significant difference.

Test Results

(1) Body Weight Change

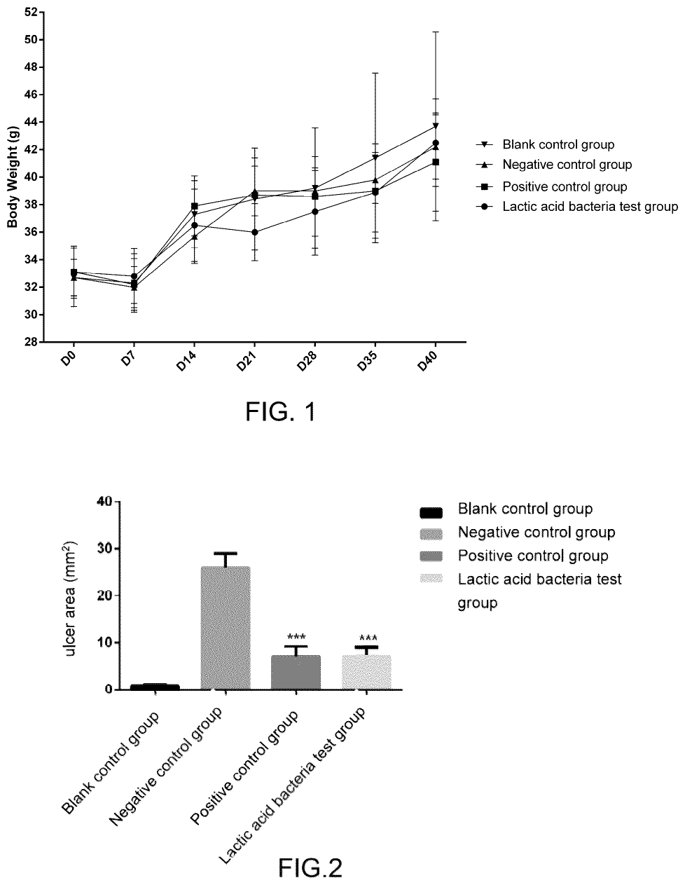

FIG. 1 shows the body weight changes of the 4 groups of mice during the test. It can be seen from FIG. 1 that there is no significant difference in the body weight of mice in each group during the same period (p>0.05). It can be seen that administering the standard drug for treating gastric ulcer (Omeprazole) and the lactic acid bacteria group of the present invention will not cause discomfort to the mice and affect the body weight change of the mice.

(2) Gastric Ulcer Area

The gastric ulcer area of each group of mice was counted according to the above-mentioned method, and the results are shown in FIG. 2. The ulcer area of the blank control group was 0.91±0.17 mm², the ulcer area of the negative control group was 26.01±2.98 mm², the ulcer area of the positive control group was 7.12±2.15 mm², and the ulcer area of the lactic acid bacteria test group was 7.40±1.60 mm². As can be seen from FIG. 2, the gastric ulcer area of the mice administered with the lactic acid bacteria group of the present invention was significantly smaller than that of the negative control group (p<0.001) and similar to that of the positive control group, which shows that the lactic acid group of the present invention can reduce drug-induced gastric injury caused by aspirin.

(3) Gastric Ulcer Index

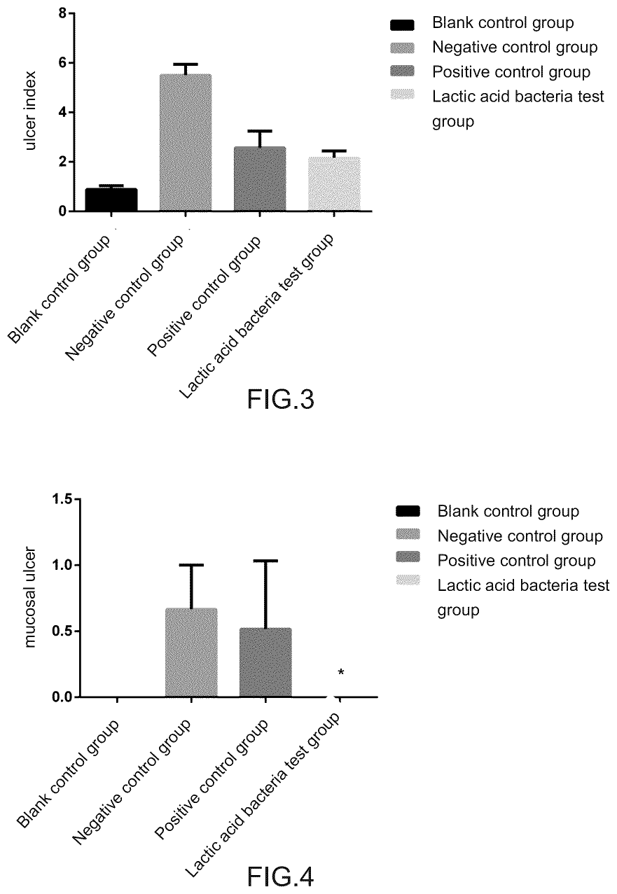

The gastric ulcer index of each group of mice was calculated according to the above-mentioned method, and the results are shown in FIG. 3. The ulcer index of the blank control group was 0.89±0.15, the ulcer index of the negative control group was 5.50±0.44, the ulcer index of the positive control group was 2.57±0.67, and the ulcer index of the lactic acid bacteria test group was 2.17±0.27. As can be seen from FIG. 3, the gastric ulcer index of the mice administered with the lactic acid bacteria group of the present invention was significantly less than that of the negative control group (p<0.001), and was also less than that of the positive control group, indicating that the lactic acid bacteria group of the present invention can reduce the drug-induced gastric damage caused by aspirin, and has even better effect than the positive control group (administration of current drugs).

(4) Cure Rate

When the test sample was administered for 4 weeks, except for the blank control group, each group was given 500 mg/kg aspirin to induce gastric ulcer symptoms for 10 days. The cure rate of each group was analyzed in Table 1 below (calculated according to the aforementioned formula 2), the cure rate of the positive control group was 53.25%, and the cure rate of the lactic acid bacteria test group was 60.61%. The cure rate of the lactic acid bacteria test group was higher than that of the positive control group using Omeprazole, showing that the lactic acid bacteria group had a better healing effect on gastric ulcers than the currently used drugs.

TABLE 1

| | | Lactic acid bacteria |
|---|---|---|
| | Positive control group | test group |
| Cure rate (%) | 53.25 | 60.61 |

(5) Stomach Pathological Analysis

The gastric tissue pathology of each group of mice was analyzed, and the pathological analysis was carried out with the aforementioned mucosal ulcer index. The analysis results of the 4 groups of mice are listed in Table 2 to Table 5 below.

TABLE 2
Mouse stomach pathological analysis of blank control group
(1) Blank control group

| Pathological | Mouse No. | | | | | |
|---|---|---|---|---|---|---|
| indicator | (1)-1 | (1)-2 | (1)-3 | (1)-4 | (1)-5 | (1)-6 |
| Mucosal ulcer | 0 | 0 | 0 | 0 | 0 | 0 |

Blank control group: no mucosal ulcer was found in histopathological analysis, and the score was 0 (the higher the score, the more serious the damage).

TABLE 3
Mouse stomach pathological analysis of negative control group
(2) Negative control group

| Pathological | Mouse No. | | | | | |
|---|---|---|---|---|---|---|
| indicator | (2)-1 | (2)-2 | (2)-3 | (2)-4 | (2)-5 | (2)-6 |
| Mucosal ulcer | 1 | 1 | 0 | 1 | 0 | 1 |

Negative control group: there were 2 rats with a degree of severity of mucosal ulcer index of 0 points, and 4 rats with a degree of severity of 1 point. It can be seen from Table 3 that the stomachs of mice in the negative control group were severely damaged.

TABLE 4
Mouse stomach pathological analysis of positive control group
(3) Positive control group

| Pathological | Mouse No. | | | | | |
|---|---|---|---|---|---|---|
| indicator | (3)-1 | (3)-2 | (3)-3 | (3)-4 | (3)-5 | (3)-6 |
| Mucosal ulcer | 0 | 0 | 1 | 0 | 1 | 0 |

Positive control group: there were 4 rats with a degree of severity of mucosal ulcer index of 0 points, and 2 rats with a degree of severity of 1 point. From the comparison of Table 3 and Table 4, it can be seen that the drug Omeprazole administered to the mice in the positive control group can effectively reduce the degree of gastric damage in the mice, especially the mucosal ulcer.

TABLE 5
Mouse stomach pathological analysis of lactic acid bacteria test group
(4) Lactic acid bacteria test group

| Pathological | Mouse No. | | | | | |
|---|---|---|---|---|---|---|
| indicator | (4)-1 | (4)-2 | (4)-3 | (4)-4 | (4)-5 | (4)-6 |
| Mucosal ulcer | 0 | 0 | 0 | 0 | 0 | 0 |

Lactic acid bacteria test group: there were 6 rats whose mucosal ulcer index severity was 0 points. From the comparison of Table 4, Table 3 and Table 2, it can be seen that the lactic acid bacteria test group can effectively reduce the degree of gastric damage in mice, and its mucosal ulcer score was significantly lower than that of the negative control group (P<0.05), and even better than that of the positive control group. It shows that the lactic acid bacteria test group of the present application can indeed reduce the gastric ulcer situation.

According to the above test results, the effective amount of the lactic acid bacteria group of the present invention based on the mouse test is 10.2 mg/kg body weight/day (10.2 mg/day per kilogram). The “effective amount” mentioned herein refers to an amount that is sufficient to produce the aforementioned preventive and/or therapeutic effects.

In a preferred embodiment, the effective amount of the active substance of the lactic acid bacteria contained in the composition is 10 mg/kg-1 g/kg body weight/day. It can be converted into a human effective dose of about 50 mg-5 g/60 kg body weight/day. This conversion method is based on “Estimating the Maximum Safe Starting Dose in Initial Clinical Trials for Therapeutics in Adult Healthy Volunteers” promulgated by the Center for Drug Evaluation and Research (CDER) of the US Food and Drug Administration (FDA). The dose conversion ratio between mice and humans is divided by 12.3, and the effective dose of humans is generally calculated with 60 kg, so 10 mg/kg body weight/day (mouse)=50 mg/60 kg body weight/day (human, 10÷ 12.3×60≈50).

In addition, if the lactic acid bacterium set of the present invention is applied to medical purposes, the following composition 1 can be used as an illustrative example.

Composition 1: The lyophilized powder (20 wt %) of Lactobacillus plantarum GKD7 and Pediococcus acidilactici GKA4, magnesium stearate (8 wt %) as a lubricant, and silicon dioxide (7 wt %) as a preservative were thoroughly mixed, dissolved in pure water (65 wt %), and stored at 4° C. for later use. The aforementioned wt % refers to the ratio of each component to the total weight of the composition.

However, although the lactic acid bacteria group of the above-mentioned embodiment is administered orally in the form of live bacteria, it can also be administered in other ways such as drops and suppositories using dead agents in practical applications.

If the lactic acid bacteria group of the present disclosure is applied to food use in a liquid dosage form, the following composition 2 is used as an illustrative example.

Composition 2: The lyophilized powder (20 wt %) of Lactobacillus plantarum GKD7 and Pediococcus acidilactici GKA4, benzyl alcohol (8 wt %) as a preservative, glycerol (7 wt %) as a diluent, and sucrose (10 wt %) as a diluent were mixed thoroughly, dissolved in pure water (55 wt %), and stored at 4° C. for later use. The aforementioned wt % refers to the ratio of each component to the total weight of the composition.

The above-mentioned embodiments are specific descriptions of several feasible embodiments of the present invention, but these embodiments are not intended to limit the present invention. Those skilled in the art may perform equivalent implementations or changes to these embodiments without departing from the technical spirit of the present invention. Therefore, the protection scope of the present invention shall be determined by the appended claims.

### BIOMATERIAL DEPOSITION

JP NITE Patent Microorganisms Depositary 2020/11/06 NITE BP-03313

JP NITE Patent Microorganisms Depositary 2020/11/06 NITE BP-03311

1. A method for treating an NSAID-induced gastric ulcer, comprising administrating an effective amount of a lactic acid bacterium set to a subject, wherein the lactic acid bacterium set comprises Lactobacillus plantarum GKD7 and Pediococcus acidilactici GKA4.

2. The method of claim 1, wherein Lactobacillus plantarum is deposited in NITE Patent Microorganisms Depositary (NPMD), Japan, and the deposit number is NITE BP-03313.

3. The method of claim 1, wherein Pediococcus acidilactici GKA4 is deposited in NITE Patent Microorganisms Depositary (NPMD), Japan, and the deposit number is NITE BP-03311.

4. The method of claim 1, wherein a weight ratio of Lactobacillus plantarum GKD7 and Pediococcus acidilactici GKA4 in the lactic acid bacterium set is 1:1.

5. The method of claim 1, wherein the effective amount of the lactic acid bacterium set is 50 mg to 5 g/60 kg body weight/day.

6. The method of claim 1, wherein treating NSAID-induced gastric ulcer refers to that a mucosal ulcer index of the subject administered with the lactic acid bacterium set is lower compared to a subject not administered with the lactic acid bacterium set.

7. The method of claim 1, wherein the lactic acid bacterium set is prepared by the following steps:
(a) inoculating bacteria of the lactic acid bacterium set into a solid-state medium for solid-state culture to form a colony;
(b) inoculating the colony cultivated in step (a) into a liquid medium culture to obtain a bacterial liquid;
(c) centrifuging the bacterial liquid obtained in step (b) to obtain a bacterial slime; and
(d) freeze-drying the bacteria slime obtained in step (c) into a freeze-dried powder;
wherein bacteria of Lactobacillus plantarum GKD7 and bacteria of Pediococcus acidilactici GKA4 are cultured separately and then mixed.

8. The method of claim 7, wherein a freeze-drying temperature in step (d) is −196 to −40° C.

9. The method of claim 1, wherein the lactic acid bacterium set is a pharmaceutical composition, and the pharmaceutical composition further comprises a pharmaceutically acceptable carrier, an excipient, a diluent or an adjuvant.

10. The method of claim 1, wherein the lactic acid bacterium set is a food additive.

11. The method of claim 1, wherein an administration mode of the lactic acid bacterium set includes oral administration, drops and suppositories.
